(19) United States
(12) Patent Application Publication
(10) Pub. No.: US 2004/0029816 A1
(43) Pub. Date: Feb. 12, 2004

(54) Hepatoprotective activity of 10-O-P-hydroxybenzoyiaucubin

(76) Inventors: Anil Prabhakar, Jammu (IN); Bishan Datt Gupta, Jammu (IN); Krishan Avtar Suri, Jammu (IN); Naresh Kumar Satti, Jammu (IN); Swadesh Malhotra, Jammu (IN); Rakesh Kamal Johri, Jammu (IN); Bupinder Singh Jaggi, Jammu (IN); Bal Krishan Chandan, Jammu (IN); Ashok Kumar Sharma, Jammu (IN); Devinder Kumar Gupta, Jammu (IN); Bal Krishan Kapahi, Jammu (IN); Kasturi Lal Bedi, Jammu (IN); Om Parkash Suri, Jammu (IN); Gulam Nabi Qazi, Jammu (IN)

Correspondence Address: MORGAN & FINNEGAN, L.L.P., 345 Park Avenue, New York, NY, 10154-0053, US

(21) Appl. No.: 10/436,423

(22) Filed: May 12, 2003

Related U.S. Application Data
(60) Provisional application No. 60/397,359, filed on Jul. 18, 2002.

(51) Int. Cl. A61K 31/7048

(52) U.S. Cl. 514/27

(57) ABSTRACT

A method for treating and/or preventing hepatic disease conditions in mammals including human beings, said method comprising the steps of administering to the mammal an effective amount of 10-O-p-hydroxybenzoylaucubin compound of Formula 1, also called Agnuside, optionally individual or in combination with one or more pharmaceutically acceptable additives.

### FIELD OF THE INVENTION

This invention relates to hepatoprotective activity of an iridoid glycoside, 10-O-phydroxybenzoylaucubin (agnuside) of the formula 1 isolated from Vitex negundo by extracting the aerial parts/whole plant with polar solvent like 95% ethanol, methanol, aqueous ethanol or water, removing fatty non polar constituents by triturating the extract with solvents such as ethylene chloride, methylene chloride, chloroform or ethyl acetate to get a fraction from which agnuside is separated by column chromatography. The hepatoprotective activity of agnuside has been confirmed by evaluation of its protective action against CCl and galactosamine induced liver damage models.

### BACKGROUND AND PRIOR ART REFERENCES

Vitex negundo Linn (family Verbenaceae) is widely used in the indigenous system of medicine in India. Various medicinal properties are ascribed to leaves and roots of this plant. The leaves are aromatic, tonic and vermifuge and the roots are used as expectorant, febrifuge and tonic.(Chopra, R. N., Nayar, S. L. and Chopra, I. C., Glossary of Indian Medicinal Plants, CSIR, New Delhi, 1956, p. 256; Wealth of India : Raw Material, CSIR, New Delhi, 1976, vol. X. p. 522 ) A number of compounds have been isolated from various parts of this plant From the leaves Ghosh and Krishna isolated a number of compounds viz. glucononitol, p-hydroxybenzoic acid, 5-hydroxyisophthalic acid and′ 3,4-dihydroxybenzoic acid along with two glucosides and an amorphous alkaloid [Ghosh, T. P. and Krishna, S.,7. Indian Chem. Soc. 1936, 13, 634]. Masilungan reported the presence of the terpenes, oc-pinene, camphene, citral and β-caryophyllene in the essential oil of the leaves Masilungan, V. A. Philip. J. Sci. 1955, 84, 275]. A large number of flavonoids have been isolated from the leaves and twigs. These are casticin, orientin, isoorientin, luteolin, luteoIin-7-0-glucoside, corymbosin, gardenins A and B, 3-0-desmethylartemetin,5-0-desmethylnobiletin, 3′,4′,5,5′,6,7,8-heptamethoxy-flavone, 3′,5-dihydroxy-4′,7,8-trimethoxyflavanone and 3′,5-dihydroxy-4′,6,7-trimethoxy flavanone [Sirait. L. M., Rimpler, H. and Haensal, R., Experientia 1962, 18, 72; Haensal, R. et al. Phytochemistry 1965,4 ,19; Banerji A. el al. Phytochemistry 1969, 8,511; Ferdous, A. J. et al. Bangladesh Acad.Sci. 1984, 8,23; Dayrit, F. M. et al. Philipp. J.Sci. 1987, 116, 403; Banerji, J. et al. Indian J. Chem.1988; 27B, 597; Achari, B. et al. Phytochemistry. 1984, 23, 703]. Stem-bark afforded five new flavone glycosides along with luteolin and acerosin. The new flavone glycosides are 6|3-glucopyranosyl-7-hydroxy-3′,4′,5′,8-tetramethoxyflavone-5-O-a-L-rhamnopyranoside, 3′,7-dihydroxy-4′,6,8-trimethoxy flavone-5-O-(6″-O-acetyl-p-D-glucopyranoside),3,3′,4′,6,7-pentamethoxy flavone 5′-O-(4″-O-|3-D-glucopyranosyl-a-rhamnopyranoside, 4′,5,7-tri-hydroxyflavone-8-(2″-caffeoyl-a-glucopyranoside) and 3′,5,5′,7-tetrahydroxy-4-methoxyflavone-3′-O-(4″-O-a-D-galactopyranosyl) galactopyranoside [Rao, V. K. et al. Indian J. Pharm. 1977,39, 41; Subramamian, P. M. and Misra, G. S. Indian J. Chem. 1978,16B, 615; Subramaniam, P. M. and Misra, G. S. J. Nat. Prod. 1979,42, 540]. A diterpenoid, 5 |3-hydro-8,11,13-abieta-trien-6a-ol and three triterpenoids, 2a, 3a-dihydroxyoleana-5 12-dien-28-oic acid, 2a, 3a-diacetoxyoleana-5, 12-dien-28-oic acid, 2,3 cc-diacetoxy-18-hydroxyoleana-5,12-dien-28-oic acid have been isolated from the seeds. These compounds exhibited anti inflammatory activity [Chawla, A. S., Sharma, A. K., Handa, S. S. and Dhar, K. L. Indian J. Chem. 1991, 30B, 773 and J.Nat. Prod. 1992,55,163]. Leaves, however, did not yield any triterpenoids but from the roots acetyloleanolic acid was isolated [Vishnol, S. P.,Shoeb, A., Kapil, R. S. and Popli, S. P. Phytochemistry 1983, 22, 597].

Five Iridoid glycosides have been reported from the leaves of V. negundo. These are aucubin, agnuside (Hansal et al. Phytochemistry 4, 1965, 9 negundoside 6′-p-hydroxybenzoyl mussaenosidic acid (Sehgal et al. Phytochemistry, 21, 1982, 363) and nishindaside (Datta et al. Tetrahedron 39,1983, 3067).

During our search for hepatoprotectives agents of plant origin, the aqueous alcoholic extract of V. negundo and a fraction isolated from it exhibited strong immuno-stimulating hepatoprotective activities. A process has been developed for the isolation of an immuno-stimulating agent from the leaves of Vitex negundo for which a patent has been granted to Regional Research Laboratory, Jammu [Suri, J. L. et. al Indian Patent No. 78388 dt. 19-03.97). Another patent application has been submitted by Regional Research Laboratory, Jammu for a process for isolation of a bioactive composition possessing hepatoprotective and immuno-stimulating activity (Application no. 16/DEL98 dt. 16.1.98) In view of the strong hepatoprotective activity exhibited by the iridoid glycosides of Picrorhiza kurroa (Ansari, R. A. et al Indian J. Med. Research, 1988, 87, 401) it was thought desirable to evaluate the iridoid glycosides of V. negundo for hepatoprotective activity. Agnuside, an iridoid glycoside, was isolated from V. negundo and evaluated for hepatoprotective activity alongwith the aqueous alcoholic extract of the plant. Both aqueous alcoholic extract (coded as 033) and agnuside (coded as 033 (1) showed marked hepatoprotective activity in experimentally induced hepatic damage with CCl₄ and galactosamine (GalN) in rats. A comparison with the known hepatoprotective agent silymarin revealed that 033 and 033 (1) exhibited higher hepatoprotective potential in most of the parameters with respect to their effect on elevated levels of serum and liver homogenate parameters (Table 1 and 2).

### DESCRIPTION OF THE INVENTION

Thus the main objective of the present invention is to provide hepatopro tec live activity of a defined bioactive molecule isolated from leaves of V. negundo viz., agnuside of formula 1, as shown in the diagram accompanying this specifications. Accordingly, the present invention provides hepatoprotective activity of a compound of formula 1 accompanying the specifications which comprises

(a) powdering the plant material by known methods

(b) preparing the aqueous alcoholic extract by percolation

(c) concentrating the alcoholic extract by conventional method,

(d) removing fatty non polar constituenls by triturating the extract with solvents such as ethylene chloride, methylene chloride, chloroform or ethyl acetate

(e) adsorbing the residue extract over silica gel,

(f) isolation of agnuside from the adsorbed extract by column chromatography and

(g) evaluating for hepatoprotective activity

The solvent used for extraction in step (b) is ethanol, aqueous ethanol, methanol, aqueous methanol or water.

The hepatoprotective activity of the compound is expressed in 50 mg/kg^{31 1} oral dose in rals which on extrapolation comes to be 300-4QO mg daily human dose (70 kg) in single or divided doses.

### CHARACTERISATION OF AGNUSIDE 1

1 obtained as crystalline compound, mp 148-50° C. IvT: −466, UV-232 nm, IR (KBr) spectrum showed absorptions at 3400, 1700, 1642 and 1618 cm^{31 1}. ¹H NMR (200 MHz, CD₃OD) 5 6.35 {dd, J2,6, H-3) 5.12 (dd, J, 4,6, H-4) 2.70 (m-H-5) 4.48 (m, H-6) 5.82(s, H-7) 2.94 (m, H-9) 5.05 (s, H-10) 4.69 (d, J 8, H-1′) 3.65 (m, H-2′) 7.92 fdd,/2,7,H-2 ″6″) 6.84 (dd, 7,2,7, H-3 ″) ¹³CNMR δ 97.92(C-1), 141.50 (C-3), 105.35 (C-4), 46.04 (C-5) 82.58 (C-6) 132.09 (C-7), 142.55 (C-8), 46.04 (C-9) 63.46 (C-10), 100.07 (C-1′) 74.56 (C-2′), 77.61 (C-3′) 71.01 (C-4′), 77.82 (C-5′)62.49 (C-6′) 121.81 (C-1″) 132.73 (C-2″,C-6″), 16.08 (C-3″, 5″), 163.50 (C-4″), 167.70 (CO)

### DETAILED DESCRIPTION OF THE INVENTION

Accordingly, the present invention provides a method for treating and/or preventing hepatic disease conditions in mammals including human beings, said method comprising the steps of administering to the mammal an effective amount of 10-O-p-hydroxybenzoylaucubin compound of Formula 1, also called Agnuside, optionally individual or in combination with one or more pharmaceutically acceptable additives.

Another embodiment of the present invention wherein the said composition reduces the elevated levels of serum glutamin-pyruvic transaminase (GPT) about 70%.

In yet another embodiment of the present invention wherein the said composition reduces the elevated levels of serum glutamin-oxalo acetic transaminase (GOT) about 60%.

In still another embodiment of the present invention wherein the said composition reduces the elevated levels of serum alkaline phosphatase (ALP) about 62%.

In yet another embodiment of the present invention wherein the said composition reduces the elevated levels of serum tryglycerides about 70%.

In still another embodiment of the present invention wherein said composition against the elevated level of bilirubin about 72%.

In yet another embodiment of the present invention wherein compound agnuside is obtained from the whole plant.

In yet another embodiment of the present invention wherein 10-O-p-hydroxybenzoylaucubin is of concentration ranging between about 20 to 200 mg/kg-body weight.

In still another embodiment of the present invention wherein 10-O-p-hydroxybenzoylaucubin is of concentration is about 50 mg/kg-body weight.

In yet another embodiment of the present invention wherein the pathological condition is selected from liver disorder

In still another embodiment of the present invention wherein the subject is selected from mammals and animals preferably humans.

In yet another embodiment of the present invention wherein said composition is used singly or in combination with pharmaceutically acceptable carriers.

In still another embodiment of the present invention wherein said composition is administered to subject in combination with pharmaceutically acceptable additives, carriers, diluents, solvents, filters. Lubricants, excipients, binder or stabilizers.

In yet another embodiment of the present invention wherein the desired dosage is administered for both preventive and curative properties.

In still another embodiment of the present invention wherein said composition is administered, orally or by any clinically/medically accepted methods.

In yet another embodiment of the present invention wherein the preferred dosage for human beings is about 5 mg/Kg of body weight.

In still another embodiment of the present invention wherein the various physical forms in which the composition is available, e.g. powder, tablet, capsule, syrup, granules, emulsion, aerosoal, or beads.

In yet another embodiment of the present invention is useful for Liver cirrhosis, Galactosemia, Hemoanigoma, Hemochromatosis, Hepatitis A, Hepatitis B, Hepatitis C, Hepatitis D, Hepatitis E, Hepatitis G, Alcholic Liver disease, Autoimmune hepatitis, Cancer of Liver, Biliary Atresia, Glycogen Storage Disease 1, Alpha-1-antitrypsin deficiency, Alagille syndrome, Byler Disease, Caroli disease, Fatty liver, Itching in Liver, Primar Biliary Cirrhosis, Sclerosing Cholangitis or Protoporphyria Erythroepatic.

One more embodiment of the present invention a process for the isolation of compound 1-O-p-hydroxybenzoylaucubin compound of Formula 1, also called Agnuside, said compound isolated from aerial part/whole body comprising of steps:

(a) powdering the plant material by known methods

(b) preparing the aqueous alcoholic extract by percolation

(c) concentrating the alcoholic extract by conventional method

(d) removing fatty non-polar constituents by triturating the extract with solvents such as ethylene chloride, methylene chloride, chloroform or ethyl acetate

(e) adsorbing the residue extract over silica gel

(f) isolating of 2′-p-Hydroxy benzoyl mussaenosidic acid from the adsorbed extract by column chromatography

### BRIEF DESCRIPTION OF THE ACCOMPANYING DRAWRINGS

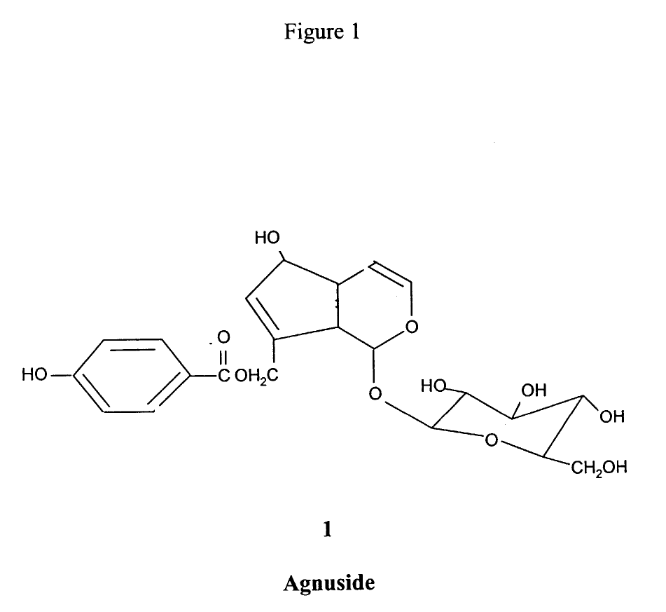

FIG. 1 represents the compound 10-O-p-hydroxybenzoylaucubin (Agnuside) of formula 1.

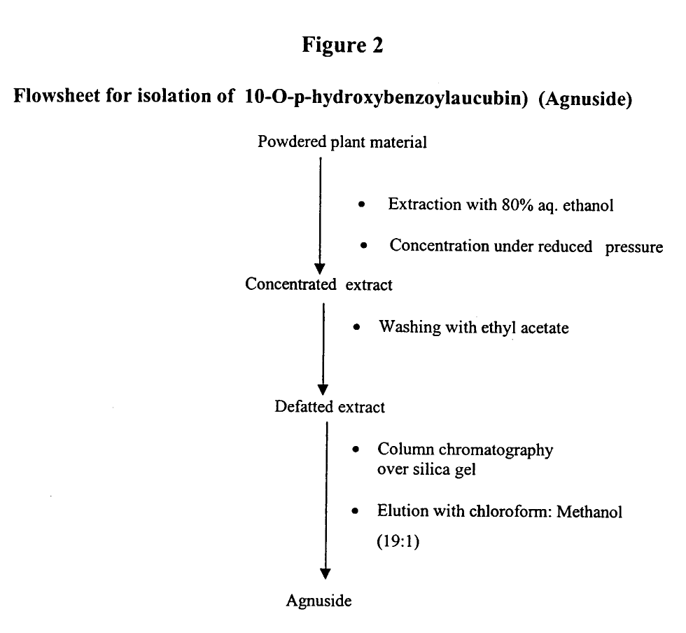

FIG. 2 represents flow-sheet for isolation of 10-O-p-hydroxybenzoylaucubin) (Agnuside).

The invention is described in detail by the examples given below which should not be construed to the limit of scope of the present invention

### EXAMPLE 1

The shade dried and powdered leaves of (1 kg) V. negundo were extracted with 80% ethanol (4×3L) by percolation. The pooled extract was concentrated under reduced pressure at below 50° C. to 1 litre of aqueous concentrate. The aqueous concentrate was washed with ethyl acetate (3×500 ml) and further concentrated to 400 ml. This syrupy residue was adsorbed over silica gel (400 g) and allowed to dry at room temperature. The slurry was put on silica gel column and eluted with mixture of chloroform-methanol (19:1), furnished 1 (2.1 g) (coded as 033 (1)

### EXAMLPLE 2

The finely ground leaves (200 g) of Vitex negundo were extracted thrice with petroleum ether (60-80°) (1 litre for the first extraction and 600 ml each for two subsequent×extractions) The drug was freed of solvent at room temperature. It was then extracted with ethanol (1 litre for the first extraction and then thrice with the same solvent 600 ml each time) Evaporation of ethanol in a rotary film evaporator yielded 30 g of extract (coded as

### EXAMPLE 3

Treatment of experimental animals with the 033 and 033(1) against CCU induced liver damage reduced the elevated levels of serum GPT, GOT, ALP, bilirubin, TG and hepatic lipid peroxidation and increased the GSH level. A comparison with the known hepatoprotective agent silymarin revealed that 033 and 033(1) exhibited higher protective potential in most of the parameters with respect to their effect on elevated levels of above parameters by CCU The hepatoprotective activity observed with 033 and 033(1} was: serum GPT-67.52 & 59.52, GOT-58.91 & 57.42, ALP-58.26 & 60.17, Bilirubin-60.00 & 71.79, TG-46.47 & 38.53 and in liver homogenate LP-55.71 & 41.48 and GSH-61.42 & 38.12% respectively. The same with silymarin was: 58.44, 51.63, 52.26, 57.50, 41.30, 62.05 & 60.15 respectively (Table-1).

### EXAMPLE 4

Treatment of animals against the galactosamine (GaIN) induced hepatic damage also reduced the elevated levels of serum GPT, GOT, ALP, bilirubin, TG and hepatic lipid peroxidation and increased the GSH level . The hepatoprotective activity observed with 033 and 033(1) was 59.58 & 44.97, 56.62 & 44.05, 54.28 & 47.66, 57.71 & 66.66, 57.89 & 49.33 percent in serum GPT, GOT, Bilirubin, ALP, and TG respectively and 67.51 & 48.34, 66.56 & 55.13 percent in hepatic lipid peroxidation (LP) and GSH respectively. The same with silymarin was 57.38, 55.40, 60.00, 53.54,46.17, 69.59, 67.18 percent respectively (Table-2).

Advantages of the present invention over currently used plant based hepatoprotectives:

1. Agnuside is more potent than the commercially available herbal hepatoprotective agent silymarin.

2. Silymarin is a mixture of three constituents whose relative proportion varies from batch to batch while agnuside is a pure compound.

TABLE 1
Hepatoprotective activity (in vivo) of 033, 033 (1) and Silymarin fed at 48 h, 24 h,
2 h before and 6 h after CCU (1 ml/kg, p.o.) induced hepatic injury in rats³.

| | Dose | Serum parameters | | | | | Hepatic parameters | |
|---|---|---|---|---|---|---|---|---|
| | mg/kg | OPT | | | Bihrubin | Triglycerides | Lipid | |
| Treatment | p. o. | (Units) | GOT (Units) | Al.F | (mg %) | (mₛ %) | Peroxidation^{c} | Glutathione^{d} |
| 033 + CCl₄ | 400 | 67.52 | 58.91 | 58.26 | 60.00 | 46.47 | 55.71 | 61.42 |
| 033 (1) + | 50 | 59.52 | 57.43 | 6017 | 71.79 | 38.53 | 41.48 | 38.12 |
| CCl₄ | | | | | | | | |
| Silymarin + | 50 | 58.44 | 51.63 | 52.26 | 57.50 | 41.30 | 62.05 | 60.15 |
| CCl₄ | | | | | | | | |

^{a}Values represent the mean percent hepatoprotective activity of six animals in each group.
H: Hepatoprotective activity was calculated as {I − (T − V/C · V)} × 100 where ‘T’ is mean value of drug and CCU,
“C” is mean value of CCU alone and “V” is the mean value of vehicle treated animals.
Unit: each unit is (mole pyruvate/min/L.
^{b}is 11 mole of p-nitrophenol formed/mini U
^{c}is n moles MDA/g liver.,
^{d}is n mole GSH/g liver

TABLE 2
Hepatoproteclive activity (m vivo) of 033, 033 (1) and Silymarin fed at 48 h,
24 h, 2 h before and 6 h after GaIN (300 mg/kg, s.c.) induced hepatic injury in rats^{a}.

| | | Serum parameters | | | | | Hepatic parameters | |
|---|---|---|---|---|---|---|---|---|
| | Dose | GPT | GOT | Bilirubin | | Triglycerides | Lipid | Glulathi |
| Treatment | mg/kg, p.o | (Units) | (Units) | (mg %) | ALP^{b} | (mg %) | peroxidation^{c} | One^{d} |
| 033 + GaIN | 400 | 59.58 | 56.62 | 54.28 | 57.71 | 57.89 | 67.51 | 66.56 |
| 033 (1) + GaIN | 50 | 44.97 | 44.05 | 47.66 | 66.66 | 49.33 | 48.34 | 55.13 |
| Silymarirri- | 50 | 57.38 | 55.40 | 60.00 | 53.54 | 46.17 | 69.59 | 67.18 |
| GaIN | | | | | | | | |

^{a}Values represent the mean percent hepatoproteclive activity of six animals in each group.
H: Hepatoprotective activity was calculated as {I − (T − V/C − V)} × 100 where “T” is mean value of drug and GaIN, ’C' is mean value of GaIN alone and “V” is the mean value of vehicle treated animals.
Unit: each unit is pinole pyruvate/min/L.
^{b}is M, mole of/j-nitrophenol formed/min/L,
^{c}is n moles MDA/g liver.,
^{d}is p. mole GSH/g liver.,

What is claimed:

1. A method for treating and/or preventing hepatic disease conditions in mammals including human beings, said method comprising the steps of administering to the mammal an effective amount of 10-O-p-hydroxybenzoylaucubin compound of Formula 1, also called Agnuside, optionally individual or in combination with one or more pharmaceutically acceptable additives.

2. A method as claimed in claim 1, wherein the said composition reduces the elevated levels of serum glutamin-pyruvic transaminase (GPT) about 70%.

3. A method as claimed in claim 1, wherein the said composition reduces the elevated levels of serum glutamin-oxalo acetic transaminase (GOT) about 60%

4. A method as claimed in claim 1, wherein the said composition reduces the elevated levels of serum alkaline phosphatase (ALP) about 62%.

5. A method as claimed in claim 1, wherein the said composition reduces the elevated levels of serum tryglycerides about 70%.

6. A method as claimed in claim 1, wherein said composition against the elevated level of bilirubin about 72%.

7. A method as claimed in claim 1, wherein compound agnuside is obtained from the whole plant.

8. A method as claimed in claim 1, wherein 10-O-p-hydroxybenzoylaucubin is of concentration ranging between about 20 to 200 mg/kg-body weight.

9. A method as claimed in claim 1, wherein 10-O-p-hydroxybenzoylaucubin is of concentration is about 50 mg/kg-body weight.

10. A method as claimed in claim 1, wherein the pathological condition is selected from liver disorder

11. A method as claimed in claim 1, wherein the subject is selected from mammals and animals preferably humans.

12. A method as claimed in claim 1, wherein said composition is used singly or in combination with pharmaceutically acceptable carriers.

13. A method according to claim 1 wherein said composition is administered to subject in combination with pharmaceutically acceptable additives, carriers, diluents, solvents, filters. Lubricants, excipients, binder or stabilizers.

14. A method as claimed in claim 1, wherein the desired dosage is administered for both preventive and curative properties.

15. A method as claimed in claim 1, wherein said composition is administered, orally or by any clinically/medically accepted methods.

16. A method as claimed in claim 1, wherein the preferred dosage for human beings is about 5 mg/Kg of body weight.

17. State the various physical forms in which the composition is available, e.g. powder, tablet, capsule, syrup, granules, emulsion, aerosoal, or beads.

18. A method as claimed in claim 1, is useful for Liver cirrhosis, Galactosemia, Hemoanigoma, Hemochromatosis, Hepatitis A, Hepatitis B, Hepatitis C, Hepatitis D, Hepatitis E, Hepatitis G, Alcholic Liver disease, Autoimmune hepatitis, Cancer of Liver, Biliary Atresia, Glycogen Storage Disease 1, Alpha-1-antitrypsin deficiency, Alagille syndrome, Byler Disease, Caroli disease, Fatty liver, Itching in Liver, Primar Biliary Cirrhosis, Sclerosing Cholangitis or Protoporphyria Erythroepatic.

19. A process for the isolation of compound 10-O-p-hydroxybenzoylaucubin compound of Formula 1, also called Agnuside, said compound isolated from aerial part/whole body comprising of steps:
1. powdering the plant material by known methods
2. preparing the aqueous alcoholic extract by percolation
3. concentrating the alcoholic extract by conventional method
4. removing fatty non-polar constituents by triturating the extract with solvents such as ethylene chloride, methylene chloride, chloroform or ethyl acetate
5. adsorbing the residue extract over silica gel
6. isolating of 2′-p-Hydroxy benzoyl mussaenosidic acid from the adsorbed extract by column chromatography
